(19) United States
(12) Patent Application Publication
(10) Pub. No.: US 2023/0414110 A1
(43) Pub. Date: Dec. 28, 2023

(54) Body Temperature Estimation Device

(71) Applicant: Nippon Telegraph and Telephone Corporation, Tokyo (JP)

(72) Inventors: Shin Toyota, Tokyo (JP); Kazuyoshi Ono, Tokyo (JP)

(21) Appl. No.: 18/254,703

(22) PCT Filed: Dec. 2, 2020
(86) PCT No.: PCT/JP2020/044811

§ 371 Date: May 26, 2023

(51) Int. Cl. A61B 5/01; G01J 5/48

(52) U.S. Cl. CPC A61B 5/01; G01J 5/48

(57) ABSTRACT

A holding mechanism is worn on the head of a person to be measured. An infrared sensor is provided in the holding mechanism and measures infrared radiation emitted from the surface of the face of the person to be measured. The infrared sensor two-dimensionally measures, for example, the distribution of infrared radiation emitted from the surface of the face including an inner corner portion of the eye of the person to be measured. An estimation unit estimates a body temperature of the person to be measured from a measurement result of the infrared sensor. A display control unit displays the body temperature estimated by the estimation unit on a display unit.

### CROSS-REFERENCE TO RELATED APPLICATIONS

This application is a national phase entry of PCT Application No. JP/JP2020/044811, filed on Dec. 2, 2020, which application is hereby incorporated herein by reference.

### TECHNICAL FIELD

The present invention relates to a body temperature estimation device.

### BACKGROUND

There is known a body temperature measuring device that captures a thermal image of a person to be measured using infrared thermography (a thermal image sensor) and estimates a body temperature such as a core body temperature of the person to be measured from the obtained thermal image. This body temperature measuring device is a convenient measuring device because the body temperature can be measured instantaneously without contact with the person to be measured.

All objects radiate energy of a certain wavelength by vibration or rotation of atoms or molecules as long as the energy is absolute zero (0 K: −273.15° C.) or more. Infrared thermography receives energy radiated from an object and obtains a temperature of the object from Stefan-Boltzmann's law, thereby imaging the object as a two-dimensional temperature distribution.

The infrared thermography having such features is applied in a wide range of fields such as quality control, plant maintenance, structural diagnosis, and security monitoring of industrial products as well as in the electric and electronic fields. As described in Non Patent Literature 1, one application example of this technology is a pandemic countermeasure, and it is possible to prevent the spread of infection by sensing heat generation due to an infectious disease such as influenza by installing the technology in a gate of an airport or the like.

However, in a case where the infrared thermography is used for the body temperature measurement in a situation where the flow line is not fixed such as heat stroke countermeasures during exercise, there is a problem that a rise in the body temperature of the person to be measured cannot be detected because the person to be measured is moving. Although there is also a technology of monitoring the body temperature of the person to be measured during exercise using a contact type temperature sensor, a decrease in air permeability of the contact portion and a touch of the temperature sensor may cause not only psychological discomfort but also physical damage such as eczema depending on a person.

### CITATION LIST

### Non Patent Literature

Non Patent Literature 1: Jiro Ota and Eri Hamada, “Environmental Technology Utilized in Society: An Introduction to Case Studies of Body Surface Temperature Measurement Using Infrared Thermography from the Perspective of Preventing the Spread of Influenza”, NEC Technical Journal, vol. 62, no. 3, pp. 87-91, 2009.

### SUMMARY

### Technical Problem

As described above, in a case where the infrared thermography is used to measure the body temperature of the person to be measured in a situation where the flow line is not fixed such as exercise, there is a problem that a change in the body temperature such as a rise in the body temperature of the person to be measured cannot be measured.

Embodiments of the present invention have been made to solve the above problems, and an object of embodiments of the present invention is to enable measurement of a change in a body temperature of a person to be measured even in a situation where a flow line is not fixed.

### Solution to Problem

A body temperature estimation device according to embodiments of the present invention includes: a holding mechanism that is worn on the head of a person to be measured; an infrared sensor that is provided in the holding mechanism and measures infrared radiation emitted from a surface of the face of the person to be measured; an estimation unit that estimates a body temperature of the person to be measured from a measurement result of the infrared sensor; and a display control unit that displays the body temperature estimated by the estimation unit on a display unit.

### Advantageous Effects of Embodiments of Invention

As described above, according to embodiments of the present invention, since the holding mechanism is provided with the infrared sensor that measures infrared radiation emitted from the surface of the face of the person to be measured, it is possible to measure a change in the body temperature of the person to be measured even in a situation where the flow line is not fixed.

### BRIEF DESCRIPTION OF THE DRAWINGS

FIG. 1 is a configuration diagram illustrating a configuration of a body temperature estimation device according to an embodiment of the present invention.

FIG. 2 is a perspective view illustrating a more detailed configuration of the body temperature estimation device according to the embodiment of the present invention.

FIG. 3 is a configuration diagram illustrating a partial configuration of the body temperature estimation device according to the embodiment of the present invention.

FIG. 4 is a configuration diagram illustrating a partial configuration of the body temperature estimation device according to the embodiment of the present invention.

FIG. 5 is a distribution diagram illustrating a temperature distribution of a surface measured from a left side of a face.

FIG. 6A is a distribution diagram illustrating a temperature distribution of a surface measured from a front left side of a face.

FIG. 6B is a distribution diagram illustrating a temperature distribution of a surface measured from a front right side of the face.

### DETAILED DESCRIPTION OF ILLUSTRATIVE EMBODIMENTS

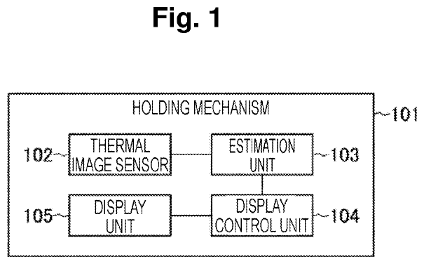

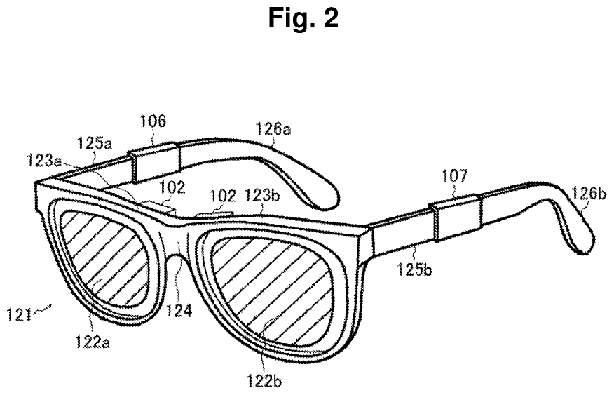

A body temperature estimation device according to an embodiment of the present invention will be described below with reference to FIGS. 1 and 2. The body temperature estimation device includes a holding mechanism 101, an infrared sensor 102, an estimation unit 103, a display control unit 104, and a display unit 105.

The holding mechanism 101 is worn on the head of a person to be measured. The holding mechanism 101 can include, for example, a face guard or a mouth shield. The infrared sensor 102 is provided in the holding mechanism 101 and measures infrared radiation emitted from the surface of the face of the person to be measured. The infrared sensor 102 can be, for example, a thermal image sensor such as an infrared thermographic camera that two-dimensionally measures the distribution of infrared radiation emitted from the surface of the face including the inner corner portion of the eye of the person to be measured.

The estimation unit 103 estimates a body temperature of the person to be measured from a measurement result of the infrared sensor 102. For example, the temperature is calculated from infrared light intensity data measured by the infrared sensor 102 to estimate the body temperature of the person to be measured. The infrared sensor 102 can also be provided at a plurality of locations, and in this case, a temperature corresponding to each of the installed locations can be calculated and obtained. Furthermore, a value obtained by averaging a plurality of temperatures derived from the results of measurement by the plurality of infrared sensors 102 can be used as the body temperature.

When the infrared sensor 102 two-dimensionally measures the distribution of infrared radiation emitted from the surface of the face including the inner corner portion of the eye of the person to be measured, the estimation unit 103 obtains a temperature distribution from the data of the light intensity distribution of infrared radiation and estimates the body temperature of the person to be measured from the obtained temperature distribution. For example, the estimation unit 103 can estimate a value obtained by averaging the obtained temperature distribution as the body temperature.

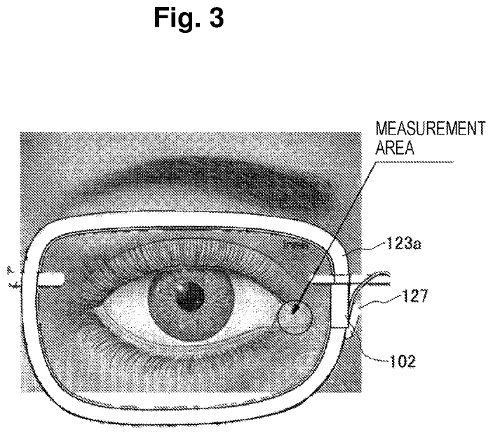

For example, the estimation unit 103 can estimate the body temperature of the person to be measured from the highest temperature in the obtained temperature distribution. In the temperature distribution obtained by measuring the surface of the face including the inner corner portion of the eye, the area having the highest temperature can be estimated as the inner corner portion of the eye. For example, as illustrated in FIG. 3, by disposing the infrared sensor 102 on a rim 123a near a pad 127, the temperature of the inner corner of the eye can be acquired. In the inner corner of the eye on the face, a pink thin film called “semilunar fold” exists. Since the semilunar fold is not covered with the skin and blood vessels pass through the semilunar fold, the semilunar fold is considered to be a place where the internal body temperature is most easily reflected when the surface temperature distribution of the face is measured. Therefore, the value of the body temperature can be obtained (estimated) by measuring the temperature of the inner corner of the eye.

The estimation unit 103 is a microcomputer including a central processing unit (CPU), a main storage device, an external storage device, a network connection device, and the like, and can also implement each of the above-described functions by causing the CPU to operate (execute the program) according to the program loaded in the main storage device. The functions can be distributed to a plurality of microcomputers.

The display control unit 104 displays the body temperature estimated by the estimation unit 103 on the display unit 105. For example, the display control unit 104 can display one value for the body temperature estimated by the estimation unit 103. The display control unit 104 can also display a temperature distribution of the body temperature measured by the infrared sensor 102. Furthermore, the display control unit 104 can display not only the body temperature but also data acquired from other sensors (not illustrated).

As illustrated in FIG. 2, the holding mechanism 101 can include an eyeglass frame 121. The infrared sensor 102 can be provided on, for example, a rim 123a and a rim 123b of the eyeglass frame 121. For example, the infrared sensor 102 may be disposed on each of the rim 123a and the rim 123b near a bridge 124. In addition, a lens 122a and a lens 122b of the eyeglass frame 121 can be configured by a transparent liquid crystal display device to form the display unit 105.

In addition, a power supply 106 that supplies power to the infrared sensor 102, the estimation unit 103, and the display control unit 104 can be provided (built) in a temple 125a of the eyeglass frame 121. The power supply 106 can be formed with a secondary battery, for example. The power supply 106 also includes a switch that turns on and off power to be supplied. Furthermore, an arithmetic processing device 107 including the estimation unit 103, the display control unit 104, and the like can be provided (built) in a temple 125b.

Although not illustrated, power supply wiring to the lens 122a and the lens 122b configured by the power supply 106, the arithmetic processing device 107, and the transparent liquid crystal display device, signal wiring connecting between the infrared sensor 102 and the arithmetic processing device 107, and the like are built in the temple 125a, the temple 125b, the rim 123a, the rim 123b, and the bridge 124.

Furthermore, it is also possible to further include a temperature sensor that is provided so as to be contactable with the skin of the person to be measured at the portions of a temple tip 126a and a temple tip 126b of the eyeglass frame 121 and measures the temperature of the skin in contact with the temperature sensor. By using this temperature sensor, the estimation unit 103 can estimate the body temperature of the person to be measured based on a measurement result of the temperature sensor in addition to the measurement result of the infrared sensor 102. The ear portions on which the temple tip 126a and the temple tip 126b are hung are often covered with hair, and it may be difficult for the temperature sensor to directly contact the body surface. However, due to the structure of the eyeglass frame 121, by providing the above-described temperature sensors on the temple tip 126a and the temple tip 126b, the temperature sensors are configured to be pressed against the skin, thereby making it difficult for the temperature sensors to move, and enabling a stable environment measurement. In addition, the superficial temporal artery flows in the auricle, which is a portion strongly affected by heat transport due to blood flow. Therefore, by separating the temperature of the hair from the temperature of the body surface, the body temperature of the person to be measured to which the holding mechanism 101 (eyeglass frame 121) is worn can be measured.

Meanwhile, in a case where the infrared sensor 102 is provided in front of the face, it is desirable that there be a certain distance (in cm) between the infrared sensor 102 and the body surface in order to stabilize the infrared measurement being emitted. In a case where the infrared sensor 102 is provided in front, it is not easy to take the above distance, and there is also a likelihood that the infrared sensor will obstruct the view.

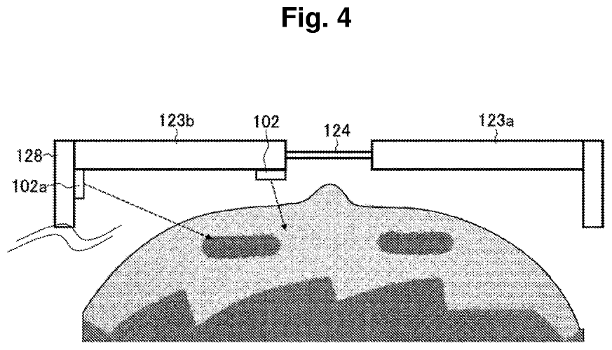

In contrast to the above, as illustrated in FIG. 4, an infrared sensor 102a is disposed on the side surface side of the face in an end piece 128 extending from the end portion of the rim 123a toward the auricle, and the measurement is performed from the oblique side of the face, whereby the distance between the infrared sensor 102a and the inner corner of the eye can be taken, and the accurate temperature can be acquired. However, depending on the disposition of the infrared sensor 102a, the temperature of the inner corner of the eye cannot be acquired. An infrared sensor can be disposed on each of the left and right end pieces.

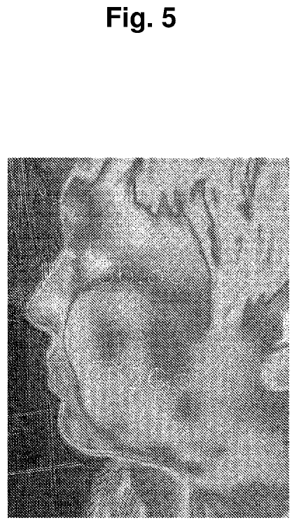

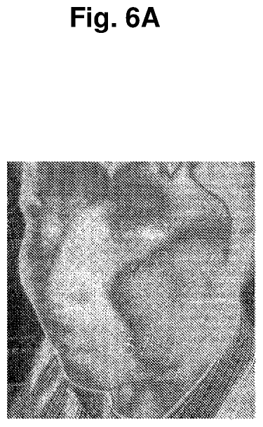

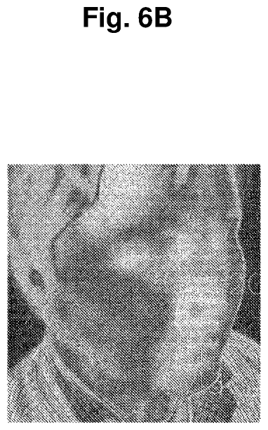

For example, when the infrared sensor 102a is disposed too close to the temple tip side, the position of the inner corner of the eye is outside the measurement area, and the temperature distribution around the rim of the eye is measured, as illustrated in the temperature distribution on the left side of the face in FIG. 5. In this case, the portion having the highest temperature is the rim of the eye. On the other hand, when the infrared sensor 102a is disposed at a position close to the boundary between the end piece 128 and the rim 123a, the temperature distribution in the area including the inner corner of the eye can be measured, as illustrated in the temperature distribution on the left side of the face in FIG. 6A. The temperature of the rim of the eye, which is the highest temperature from the temperature distribution illustrated in FIG. 5, is 36.8° C. On the other hand, the temperature of the inner corner of the eye, which is the highest temperature from the temperature distribution illustrated in FIG. 6A, is 37.2° C. There is a difference of 0.4° C. between them.

Thus, as illustrated in FIG. 4, the infrared sensor 102a is desirably disposed to be able to measure the inner corner of the eye from an oblique direction. When the measurement is performed with this configuration, as illustrated in FIG. 6A, the temperature at the position of the left inner corner of the eye is 37.2° C., which is the highest. From this measurement result, the body temperature of the person to be measured can be estimated to be 37.2° C. Further, as illustrated in the temperature distribution on the right side of the face in FIG. 6B, when there is a difference between the temperature of the left inner corner of the eye and the temperature of the right inner corner of the eye, an average value thereof can be estimated as the body temperature. When the temperature difference between them is equal to or less than a certain value, the temperature on the high temperature side can also be estimated as the body temperature. In this way, various methods can be used to estimate the temperature.

The temperature of the rim of the eye can also be used for estimation. For example, in a case where the temperature of the rim of the eye+0.5° C. is set as a threshold value of the estimated value and the temperature equal to or higher than this threshold value is calculated, the result may be regarded as a measurement error and may not be displayed.

As described above, according to embodiments of the present invention, since the holding mechanism is provided with the infrared sensor that measures infrared radiation emitted from the surface of the face of the person to be measured, it is possible to measure a change in the body temperature of the person to be measured even in a situation where the flow line is not fixed. By measuring the body temperature using a so-called wearable sensor including a holding mechanism, a change in the body temperature of the person to be measured can be measured even in a situation where the flow line is not fixed. In the configuration in which the temperature sensor comes into contact, there may be a case where psychological discomfort occurs or a problem such as eczema occurs depending on a person due to a decrease in air permeability of the contact portion or a touch of the sensor. On the other hand, when the infrared sensor is used to measure the surface temperature of the face in a non-contact manner, the above-described problem does not occur.

Note that the present invention is not limited to the embodiment described above, and it is obvious that many modifications and combinations can be implemented by a person having ordinary knowledge in the art within the technical idea of the present invention.

### REFERENCE SIGNS LIST

101 Holding mechanism
102 Infrared sensor
103 Estimation unit
104 Display control unit
105 Display unit

1-7. (canceled)

8. A body temperature estimation device comprising:
a holding mechanism configured to be worn on a head of a person to be measured;
an infrared sensor in the holding mechanism and configured to obtain a measurement result by measuring infrared radiation emitted from a surface of a face of the person to be measured;
an estimation circuit configured to estimate a body temperature of the person to be measured based on the measurement result of the infrared sensor; and
a display control circuit configured to display the body temperature estimated by the estimation circuit on a display device.

9. The body temperature estimation device according to claim 8, wherein:
the infrared sensor is configured to obtain the measurement result by two-dimensionally measuring a distribution of infrared radiation emitted from the surface of the face including infrared radiation emitted from an inner corner portion of an eye of the person to be measured; and
the estimation circuit is configured to estimate the body temperature of the person to be measured based a highest temperature among temperatures derived from the measurement result of the infrared sensor.

10. The body temperature estimation device according to claim 8, wherein:
the holding mechanism includes an eyeglass frame;
the infrared sensor is provided in the eyeglass frame; and
a lens of the eyeglass frame serves as the display device.

11. The body temperature estimation device according to claim 10, wherein:
the infrared sensor is provided on a rim of the eyeglass frame.

12. The body temperature estimation device according to claim 10, wherein:
the infrared sensor is provided on an end piece of the eyeglass frame.

13. The body temperature estimation device according to claim 12, wherein:
the eyeglass frame includes a left end piece and a right end piece; and
the infrared sensor is provided on each of the left end piece and the right end piece.

14. The body temperature estimation device according to claim 8, further comprising:
a temperature sensor provided in a temple-tip portion of an eyeglass frame configured to be contactable with skin of the person to be measured, the temperature sensor being configured to obtain a measurement result by measure a temperature of the skin in contact with the temperature sensor, wherein the estimation circuit is configured to estimate the body temperature of the person to be measured based on the measurement result of the temperature sensor in addition to the measurement result of the infrared sensor.

15. A body temperature estimation device comprising:
an eyeglass frame configured to be worn on a head of a person to be measured;
an infrared sensor in the eyeglass frame and configured to obtain a measurement result by measuring infrared radiation emitted from a surface of a face of the person to be measured;
an estimation circuit configured to estimate a body temperature of the person to be measured based on the measurement result of the infrared sensor; and
a display control circuit configured to display the body temperature estimated by the estimation circuit on a display device.

16. The body temperature estimation device according to claim 15, wherein:
the infrared sensor is configured to obtain the measurement result by two-dimensionally measuring a distribution of infrared radiation emitted from the surface of the face including infrared radiation emitted from an inner corner portion of an eye of the person to be measured; and
the estimation circuit is configured to estimate the body temperature of the person to be measured based a highest temperature among temperatures derived from the measurement result of the infrared sensor.

17. The body temperature estimation device according to claim 16, wherein:
a lens of the eyeglass frame is the display device.

18. The body temperature estimation device according to claim 15, wherein:
the infrared sensor is provided on a rim of the eyeglass frame.

19. The body temperature estimation device according to claim 15, wherein:
the infrared sensor is provided on an end piece of the eyeglass frame.

20. The body temperature estimation device according to claim 15, wherein:
the eyeglass frame includes a left end piece and a right end piece; and
the infrared sensor is provided on each of the left end piece and the right end piece.

21. The body temperature estimation device according to claim 15, further comprising:
a temperature sensor provided in a temple-tip portion of the eyeglass frame to be contactable with skin of the person to be measured, the temperature sensor being configured to obtain a measurement result by measure a temperature of the skin in contact with the temperature sensor, wherein the estimation circuit is configured to estimate the body temperature of the person to be measured based on the measurement result of the temperature sensor in addition to the measurement result of the infrared sensor.
